(19) United States
(12) Patent Application Publication
(10) Pub. No.: US 2025/0029508 A1
(43) Pub. Date: Jan. 23, 2025

(54) GROUP TRAINING ACTION CORRECTION SYSTEM AND METHOD COMBINING FACE AND GESTURE RECOGNITION

(71) Applicant: Air Force Medical Center, PLA, Beijing (CN)

(72) Inventors: Qiao Ye, Beijing (CN); Guangyun Wang, Beijing (CN); Wei Sun, Beijing (CN); Ruibo Qi, Suzhou (CN); Yuan Luo, Beijing (CN); Zhusong Mei, Beijing (CN); Bingqian Guo, Beijing (CN); Longmei Fang, Beijing (CN); Chengxiang Zhu, Suzhou (CN); Guiling Bi, Suzhou (CN); Yao Li, Suzhou (CN); Yin Zhang, Suzhou (CN)

(21) Appl. No.: 18/770,760

(22) Filed: Jul. 12, 2024

(30) Foreign Application Priority Data
Jul. 20, 2023 (CN) 202310889994.6

(51) Int. Cl. G09B 5/02; G06T 13/00; G06V 20/50; G06V 40/16; G06V 40/20

(52) U.S. Cl. CPC G09B 5/02; G06T 13/00; G06V 20/50; G06V 40/172; G06V 40/20

(57) ABSTRACT

The present disclosure provides a group training action correction system and method combining face and gesture recognition, wherein the system includes an individual action correction moment determination module, which is configured to determine a nearest individual action correction moment after a current training progress on a preset training progress axis; and an individual action correction module, which is configured to correct and prompt individually action for the action correction object based on a standard gesture, an action to be corrected, and a personnel identity when entering the individual correction moment.

### CROSS-REFERENCE TO RELATED APPLICATION

The present disclosure claims the priority to the Chinese patent application with the filing No. 2023108899946 filed with the Chinese Patent Office on Jul. 20, 2023, and entitled “GROUP TRAINING ACTION CORRECTION SYSTEM AND METHOD COMBINING FACE AND GESTURE RECOGNITION”, the contents of which are incorporated herein by reference in entirety.

### TECHNICAL FIELD

The present disclosure relates to the technical field of data recognition and processing, and particularly to a group training action correction system and method combining face and gesture recognition.

### BACKGROUND ART

At present, the action recognition correction technology is mostly realized in a one-to-one mode when applying, i.e., it plays a training action video by a teaching screen for a single trainee to view and learn to follow; recognizes an irregular training action made by the trainee; controls the teaching screen to pause the training action video; and plays a correction video of the irregular training action, so as to correct the training action for the trainee.

However, it is difficult to realize the action recognition correction technology in a one-to-many mode when applying. When multiple trainees view the training action video playing on the teaching screen, some trainees make standard follow actions, and some trainees make irregular follow actions. If the teaching screen is controlled to pause the training action video and play the correction video of the irregular training actions, the trainees who make the standard follow actions also need to stop training and view the correction video together, which affects their learning.

Therefore, a solution is urgently needed.

### SUMMARY

One purpose of the present disclosure is to provide a group training action correction system combining face and gesture recognition, which automatically generates an action to be corrected and an action correction object and determines a moment suitable for individually correcting the action to be corrected of the action correction object when an irregular training gesture produced by a trainee among multiple trainees are recognized; and corrects the corresponding action when entering the moment, which does not affect the learning of the other trainees and solves a difficulty of realizing the action recognition correction technology in one-to-many mode when applying.

The embodiments of the present disclosure provide a group training action correction system combining face and gesture recognition, including:

a training gesture recognition module, which is configured to acquire a current training progress and recognize training gestures of multiple trainees;

a gesture specification determination module, which is configured to determine whether the training gestures are standard based on a preset standard gesture corresponding to the current training progress, wherein the corresponding training gestures are taken as actions to be corrected and the trainees generating the actions to be corrected are taken as action correction objects when the training gestures are not standard;

a personnel identity determination module, which is configured to recognize face IDs of the action correction objects and acquire preset personnel identities corresponding to the face IDs;

an individual action correction moment determination module, which is configured to determine a nearest individual action correction moment after the current training progress on a preset training progress axis, wherein following operations are performed, including:

determining an action training cycle that the current training progress falls on the training progress axis;

determining a next action training cycle after the action training cycle on the training progress axis;

determining whether the next action training cycle is the same as the action training cycle,

determining a repetition training progress corresponding to the current training progress from the next action training cycle and taking as the individual action correction moment when the next action training cycle is the same as the action training cycle; otherwise, determining whether a first gap time interval exists between the action training cycle and the next action training cycle,

taking a start moment of the first gap time interval as the individual action correction moment when the first gap time interval exists between the action training cycle and the next action training cycle; otherwise, acquiring a correlation relationship between the action training cycle and the next action training cycle;

matching the correlation relationship with a triggered correlation relationship in a preset triggered correlation relationship library,

inserting a preset second gap time interval immediately after an end moment of the next action training cycle when the match exists; otherwise, inserting the second gap time interval immediately after an end moment of the action training cycle;

taking a start moment of the second gap time interval as the individual action correction moment; and

an individual action correction module, which is configured to correct and prompt individually the actions for the action correction objects based on the standard gesture, the actions to be corrected, and the personnel identities when entering the individual correction moment.

Preferably, the individual action correction module corrects and prompts individually the actions for the action correction objects based on the standard gesture, the actions to be corrected, and the personnel identities, wherein the individual action correction module performs following operations, including:

acquiring a preset first virtual action corresponding to the standard gestures and a preset second virtual action corresponding to the actions to be corrected respectively;

acquiring an action change process that the second virtual action changes to the first virtual action;

generating a demo animation for demonstrating the action change process;

acquiring a complexity of the action change process, a maximum reminder duration of the individual correction moment, and a training experience value of the action correction objects respectively;

determining play counts and a single-play duration of the demo animation based on the complexity, the maximum reminder duration, and the training experience value;

adjusting an animation duration of the demo animation to the single-play duration;

labeling the personnel identities in the demo animation; and

showing the demo animation to the action correction objects, and controlling the play counts of continuously playing the demo animation when showing.

Preferably, the individual action correction module determines play counts and a single-play duration of the demo animation based on the complexity, the maximum reminder duration, and the training experience value, wherein the individual action correction module performs following operations, including:

calculating a control value based on the complexity, the maximum reminder duration, and the training experience value, wherein a calculation formula is as follows:

$$ref = {γ}_{1} \cdot D + {γ}_{2} \cdot T + {γ}_{3} \cdot E$$

where ref is the control value, D is the complexity, T is the maximum reminder duration, E is the training experience value, and γ₁, γ₂ and γ₃ are the preset weight values;

acquiring a preset play count determination library, wherein the play count determination library includes multiple groups of one-to-one corresponding control value intervals and count terms;

determining whether the control value falls into any of the control value intervals,

taking the count terms corresponding to the control value intervals into which the control value falls as the play counts when the control value falls into the control value intervals; and

calculating the single-play duration based on the play counts and the maximum reminder duration, wherein a calculation formula is as follows:

$$t = \frac{T}{N}$$

where t is the single-play duration, T is the maximum reminder duration, and N is the play counts.

Preferably, the individual action correction module shows the demo animation to the action correction objects, wherein the individual action correction module performs following operations, including:

acquiring face positions of the action correction objects and a screen center position of a teaching screen for training and teaching beside the action correction objects respectively;

determining a straight-line distance between the face positions and the screen center position;

determining a display size requirement corresponding to the straight-line distance from a preset display size requirement library;

determining multiple free display areas that meet the display size requirements from the teaching screen;

acquiring a target face orientation of the action correction objects;

constructing a first direction vector based on the face positions and the target face orientation;

acquiring a directly faced orientation of the teaching screen;

constructing a second direction vector based on a region center position of the free display areas and the directly faced orientation;

calculating a first vector angle between the first direction vector and the second direction vector; and

suspending the demo animation on the free display area corresponding to the largest first vector angle to show, wherein

the step of acquiring the target face orientations of the action correction objects includes:

acquiring current face orientations of the action correction objects;

trying to acquire multiple desirable face directions of the action correction objects in a future preset duration,

taking the face orientation as the target face orientation when the try fails; otherwise, integrating the face orientation and the desirable face directions to acquire a face orientation set;

constructing a third direction vector and a fourth direction vector respectively based on the face position and any two face orientations in the face orientation set;

calculating a second vector angle between the third direction vector and the fourth direction vector; and

taking a direction of a sum vector of the third direction vector and the fourth direction vector of the largest second vector angle produced by calculation as the target face orientation.

The embodiments of the present disclosure provide a group training action correction method combining face and gesture recognition, including:

step S1: acquiring a current training progress and recognizing training gestures of multiple trainees;

step S2: determining whether the training gestures are standard based on a preset standard gesture corresponding to the current training progress, wherein the corresponding training gestures are taken as actions to be corrected and the trainees generating the actions to be corrected are taken as action correction objects when the training gestures are not standard;

step S3: recognizing face IDs of the action correction objects and acquiring preset personnel identities corresponding to the face IDs;

step S4: determining a nearest individual action correction moment after the current training progress on a preset training progress axis; and

step S5: correcting and prompting individually the actions for the action correction objects based on the standard gesture, the actions to be corrected, and the personnel identities when entering the individual correction moment.

Preferably, the step S4 of determining a nearest individual action correction moment after the current training progress on a preset training progress axis includes:

determining an action training cycle that the current training progress falls on the training progress axis;

determining a next action training cycle after the action training cycle on the training progress axis;

determining whether the next action training cycle is the same as the action training cycle,

determining a repetition training progress corresponding to the current training progress from the next action training cycle and taking as the individual action correction moment when the next action training cycle is the same as the action training cycle; otherwise, determining whether a first gap time interval exists between the action training cycle and the next action training cycle,

taking a start moment of the first gap time interval as the individual action correction moment when the first gap time interval exists between the action training cycle and the next action training cycle; otherwise, acquiring a correlation relationship between the action training cycle and the next action training cycle;

matching the correlation relationships with a triggered correlation relationship in a preset triggered correlation relationship library;

inserting a preset second gap time interval immediately after an end moment of the next action training cycle when the match exists; otherwise, inserting the second gap time interval immediately after an end moment of the action training cycle; and

taking a start moment of the second gap time interval as the individual action correction moment.

Preferably, the step of correcting and prompting individually the actions for the action correction object based on the standard gesture, the actions to be corrected, and the personnel identities includes:

acquiring a preset first virtual action corresponding to the standard gesture and a preset second virtual action corresponding to the actions to be corrected respectively;

acquiring an action change process that the second virtual action changes to the first virtual action;

generating a demo animation for demonstrating the action change process;

acquiring a complexity of the action change process, a maximum reminder duration of the individual correction moment, and a training experience value of the action correction objects respectively;

determining play counts and a single-play duration of the demo animations based on the complexity, the maximum reminder duration, and the training experience value;

adjusting an animation duration of the demo animation to the single-play duration;

labeling the personnel identities in the demo animation; and

showing the demo animation to the action correction objects, and controlling the play counts of continuously playing the demo animation when showing.

Preferably, the step of determining play counts and a single-play duration of the demo animations based on the complexity, the maximum reminder duration, and the training experience value includes:

calculating a control value based on the complexity, the maximum reminder duration, and the training experience value, wherein a calculation formula is as follows:

$$ref = {γ}_{1} \cdot D + {γ}_{2} \cdot T + {γ}_{3} \cdot E$$

where ref is the control value, D is the complexity, T is the maximum reminder duration, E is the training experience value, and γ₁, γ₂ and γ₃ are the preset weight values;

acquiring a preset play count determination library, wherein the play count determination library includes multiple groups of one-to-one corresponding control value intervals and count terms;

determining whether the control value falls into any of the control value intervals;

taking the count terms corresponding to the control value intervals into which the control value falls as the play counts when the control value falls into the control value intervals; and

calculating the single-play duration based on the play counts and the maximum reminder duration, wherein a calculation formula is as follows:

$$t = \frac{T}{N}$$

where t is the single-play duration, T is the maximum reminder duration, and N is the play counts.

Preferably, the step of showing the demo animation to the action correction objects includes:

acquiring face positions of the action correction objects and a screen center position of a teaching screen for training and teaching beside the action correction objects respectively;

determining a straight-line distance between the face positions and the screen center position;

determining a display size requirement corresponding to the straight-line distance from a preset display size requirement library;

determining multiple free display areas that meet the display size requirement from the teaching screen;

acquiring a target face orientation of the action correction object;

constructing a first direction vector based on the face positions and the target face orientation;

acquiring a directly faced orientation of the teaching screen;

constructing a second direction vector based on a region center position of the free display areas and the directly faced orientation;

calculating a first vector angle between the first direction vector and the second direction vector;

suspending the demo animation on the free display area corresponding to the largest first vector angle to show, wherein

the step of acquiring a target face orientation of the action correction objects includes:

acquiring current face orientations of the action correction objects;

trying to acquire multiple desirable face directions of the action correction objects in a future preset duration,

taking the face orientation as the target face orientation when the try fails; otherwise, integrating the face orientation and the desirable face directions to acquire a face orientation set;

constructing a third direction vector and a fourth direction vector respectively based on the face position and any two face orientations in the face orientation set;

calculating a second vector angle between the third direction vector and the fourth direction vector; and

taking a direction of a sum vector of the third direction vector and the fourth direction vector of the largest second vector angle produced by calculation as the target face orientation.

Other features and advantages of the present disclosure will be illustrated in the subsequent specification and partially will become apparent from the specification or will be understood by carrying out the present disclosure. The objects and other advantages of the present disclosure can be realized and obtained by structures particularly indicated in the written specification, the claims, and the drawings.

The technical solutions of the present disclosure will be described in further detail below by the drawings and embodiments.

### BRIEF DESCRIPTION OF DRAWINGS

The drawings are used to provide a further understanding of the present disclosure and constitute a part of the specification, and are used in conjunction with embodiments of the present disclosure to explain the present disclosure, which does not constitute a limitation for the present disclosure. In the drawings:

FIG. 1 shows a schematic diagram of a group training action correction system combining face and gesture recognition in the embodiments of the present disclosure; and

FIG. 2 shows a schematic diagram of a group training action correction method combining face and gesture recognition in the embodiments of the present disclosure.

### DETAILED DESCRIPTION OF EMBODIMENTS

The preferable embodiments of the present disclosure are described below in conjunction with the drawings. It should be understood that the preferable embodiments described herein are used only to illustrate and explain the present disclosure, and are not used to limit the present disclosure.

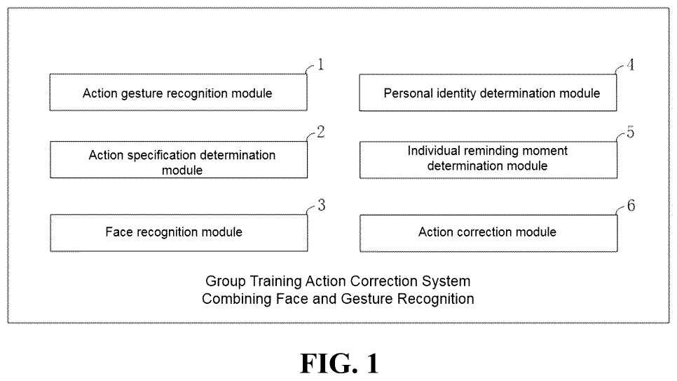

The embodiment of the present disclosure provides a group training action correction system combining face and gesture recognition, as shown in FIG. 1, including:

a training gesture recognition module 1, configured to acquire a current training progress and recognize training gestures of multiple trainees, wherein the current training progress is determined by a play progress of a training action video playing on a teaching screen; and when recognizing the training gesture of a trainee, an image of the trainee can be acquired firstly, which is realized based on an image recognition technology;

a gesture specification determination module 2, configured to determine whether the training gesture is standard based on a preset standard gesture corresponding to the current training progress, wherein the corresponding training gesture is taken as action to be corrected and the trainee generating the action to be corrected is taken as action correction object when the training gesture is not standard, wherein the standard gesture is a normative action gesture that should be produced by the trainee under the current training progress, for example, when the training action video plays to a progress of teaching an action of lying on and kicking the right leg at 90 degrees, the standard gesture is the action of kicking the right leg at 90 degrees; and when determining whether the training gesture is standard, the training gesture is compared with the standard gesture, wherein if they are not matched, the training gesture is not standard;

a personnel identity determination module 3, configured to recognize a face ID of the action correction object and acquire a preset personnel identity corresponding to the face ID, wherein the face ID is the face information, wherein a face image of the action correction object is acquired when recognizing, which is realized based on the image recognition technology; and the personnel identity can be a name, a trainee code/nickname;

an individual action correction moment determination module 4, configured to determine a nearest individual action correction moment after a current training progress on a preset training progress axis, wherein the training progress axis is a play content axis of the training action video, and the training action corresponding to play moment is labeled on the axis; and the individual action correction moment is the moment suitable for individually correcting the action to be corrected of the action correction object, and the correction at the moment does not affect the learning of the rest of the trainees; and

an individual action correction module 5, configured to correct and prompt the individual action for the action correction object based on the standard gesture, the action to be corrected, and the personnel identity when entering the individual correction moment.

The working principles and beneficial effects of the above technical solution are as follows.

The present disclosure automatically generates the action to be corrected and the action correction object and determines the moment suitable for individually correcting the action to be corrected of the action correction object when recognizing the irregular training gesture produced by the trainee among multiple trainees; and corrects the corresponding action when entering the moment, which does not affect the learning of the other trainees and solves the difficulty of realizing the action recognition correction technology in one-to-many mode when applying.

During the specific application, multiple trainees view the training action video playing on the teaching screen to learn and follow the action. When a follow action of the trainee is not standard, the training action video on the teaching screen is not controlled to be paused, and therefore all the trainees can continue to follow, which does not affect the trainees with standard follow actions to learn continuously. The system individually corrects the action for the trainee with the irregular follow action at the next nearest individual action correction moment, at which time the trainee with the irregular follow action can know his/her corresponding action and correct it.

In one embodiment, the individual action correction moment determination module 4 determines the nearest individual action correction moment after the current training progress on the preset training progress axis, wherein the individual action correction moment determination module 4 performs the following operations, including:

determining an action training cycle that the current training progress falls on the training progress axis, wherein the action training cycle is labeled on the training progress axis, and the action training cycle contains a set of actions that the trainee is required to follow continuously;

determining a next action training cycle after the action training cycle on the training progress axis;

determining whether the next action training cycle is the same as the action training cycle;

determining a repetition training progress corresponding to the current training progress from the next action training cycle and taking as the individual action correction moment when the next action training cycle is the same as the action training cycle; otherwise, determining whether a first gap time interval exists between the action training cycle and the next action training cycle, wherein when they are the same, it means that the trainee in the next action training cycle needs to repeatedly do the action to be corrected, i.e., do the action in the repetition training progress corresponding to the current training progress, in which the action correction object can be corrected at this time, so that the repetition training progress can be taken as the individual action correction moment; and a first gap time interval is labeled on the training progress axis, wherein the first gap time interval is a time interval for the trainee to rest halfway, and freely review the training action;

taking a start moment of the first gap time interval as the individual action correction moment when the first gap time interval exists between the action training cycle and the next action training cycle; otherwise, acquiring the correlation relationship between the action training cycle and the next action training cycle, wherein when existing the first gap time interval, the action of the action correction object can be corrected at the beginning, i.e., at the start moment of the first gap time interval, wherein the correlation relationship between adjacent action training cycles is labeled on the training progress axis;

matching the correlation relationship with a triggered correlation relationship in a preset triggered correlation relationship library, wherein the triggered correlation relationship is the correlation relationship indicating the action teaching that enables the action training cycle and the next action training cycle not to be able to be interrupted, for example, when the training actions taught in the action training cycle and the next action training cycle need to be followed continuously by the trainee to develop coherence of muscle memory of the trainee, the triggered correlation relationship is a coherence teaching action;

inserting a preset second gap time interval immediately after an end moment of the next action training cycle when the match exists; otherwise, inserting the second gap time interval immediately after the end moment of the action training cycle, wherein when the match exists, it means that the action teaching of the action training cycle and the next action training cycle cannot be interrupted, so that the second gap time interval is inserted immediately after the end moment of the next action training cycle (The correlation relationship between the next action training cycle and the action training cycle after next is not considered herein, i.e., whether or not the teaching actions between them can be interrupted is not considered. Since the action correction object has generated the action to be corrected, and the longer the timing of correction is delayed, the poorer the effectiveness of correction is, the importance of timely correction is much greater than the importance of whether the teaching action between them can be interrupted.); otherwise (when the match does not exist), the action correction object needs to be corrected immediately after the end of the action training cycle, i.e., the second gap time interval is inserted immediately after the end moment of the action training cycle, wherein the interval length of the second gap time interval is provided by the technician on demand; and

taking a start moment of the second gap time interval as the individual action correction moment.

The working principles and beneficial effects of the above technical solution are as follows.

The embodiment of the present disclosure, when determining the individual action correction moment, firstly determines whether the action correction object will repeat the action to be corrected immediately, and, if yes, takes the moment (the repetition training progress) when the action correction object repeats the action to be corrected as the individual action correction moment. When the action correction object does not repeat the action to be corrected, it determines whether the duration (the first gap time interval) exists for the trainee to rest halfway, and freely review the training action, etc. If it exists (generally, breaks for the trainee to rest halfway, etc., will be provided in the training action teaching video, so that it has rationality and applicability), the start moment of the duration is taken as the individual action correction moment. If it does not exist, the second gap time interval is inserted. When inserting, the correlation relationship between the action training cycle and the next action training cycle is considered, so as to determine the insertion position and reasonably insert the second gap time interval, which greatly improves the comprehensiveness, suitability, and applicability of the determination of the individual action correction moment.

In one embodiment, the individual action correction module 5 corrects and prompts the individual action for the action correction object based on the standard gesture, the action to be corrected, and the personnel identity, wherein the individual action correction module 5 performs the following operations, including:

acquiring a preset first virtual action corresponding to the standard gesture and a preset second virtual action corresponding to the action to be corrected respectively, wherein the first virtual action and the second virtual action are action animations corresponding to the standard gesture and the action to be corrected respectively;

acquiring an action change process that the second virtual action changes to the first virtual action;

generating a demo animation for demonstrating the action change process, wherein the demo animation can demonstrate to the action correction object how to change from the action to be corrected to the standard gesture;

acquiring a complexity of the action change process, a maximum reminder duration of the individual correction moment, and a training experience value of the action correction object respectively, wherein the complexity represents the difficulty that the action to be corrected changes to the standard gesture; and the greater the complexity is, the greater the difficulty is and the complexity can be provided by the technician on demand; the maximum reminder duration is divided into three cases: first, the maximum reminder duration is the total teaching duration of the repetition training progress in the training video when the individual correction moment is the repetition training progress; second, the maximum reminder duration is the interval length of the first gap time interval when the individual correction moment is the start moment of the first gap time interval; and third, the maximum reminder duration is the interval length of the second gap time interval when the individual correction moment is the start moment of the second gap time interval; and the training experience value represents the degree of experience that the action correction object learns to follow the training action; and the greater the training experience value is, the greater the degree of the experience is, wherein the training experience value can be provided by the technician according to the degree of actual experience of the trainee;

determining the play counts and the single-play duration of the demo animation based on the complexity, the maximum reminder duration, and the training experience value;

adjusting the animation duration of the demo animation to the single-play duration, wherein the content of the demo animation is not deleted when adjusting, and the play moment is adjusted by changing the play speed;

labeling the personnel identity in the demo animation, wherein the personnel identity can be labeled in each frame of the demo animation in the form of the information box when labeling; and

showing the demo animation to the action correction object, and controlling the play counts of continuous playing the demo animation when showing.

The working principles and beneficial effects of the above technical solution are as follows.

Due to the limited prompting duration (i.e., the maximum reminder duration is fixed), in order to ensure the best correction prompting effect when showing the demo animation to the action correction object, it is necessary to comprehensively determine the play counts of the demo animation. The embodiment of the present disclosure determines the play counts and the single-play duration of the demo animation based on the complexity, the maximum reminder duration, and the training experience value, which indicates reasonableness of the control of continuous play of the demo animation when showing the demo animation to the action correction object, and ensures the correction prompting effect for the action correction object when correcting the action to be corrected.

In one embodiment, the individual action correction module 5 determines the play counts and the single-play duration of the demo animation based on the complexity, the maximum reminder duration, and the training experience value, wherein the individual action correction module 5 performs the following operations, including:

calculating the control value based on the complexity, the maximum reminder duration, and the training experience value, wherein the calculation formula is as follows:

$$ref = {γ}_{1} \cdot D + {γ}_{2} \cdot T + {γ}_{3} \cdot E$$

where ref is the control value, D is the complexity, T is the maximum reminder duration, E is the training experience value, and γ₁, γ₂ and γ₃ are the preset weight values, wherein each weight value can be provided in advance by the technician on demand;

acquiring the preset play count determination library, wherein the play count determination library includes multiple groups of one-to-one corresponding control value intervals and count terms, wherein the count terms are appropriate continuous play counts of the demo animation when the control value falls into the control value interval, and the control value interval and the count terms can be provided in advance by the technician on demand, wherein the greater the complexity is and the smaller the training experience value is, in general, which means that the demo animation needs to be played more times, i.e., the continuous play counts are larger;

determining whether the control value falls into any of the control value intervals;

taking the count term corresponding to a control value interval into which the control value falls as play counts, if yes; and

calculating the single-play duration based on the play counts and the maximum reminder duration, wherein the calculation formula is as follows:

$$t = \frac{T}{N}$$

where t is the single-play duration, T is the maximum reminder duration, and N is the play counts, wherein the ratio of the maximum reminder duration to the play counts is the single-play duration.

The working principles and beneficial effects of the above technical solution are as follows.

The embodiment of the present disclosure quotes a calculation algorithm of the control value and the play count determination library to rapidly determine the play counts, which improves the efficiency of determining the play counts and the single-play duration of the demo animation.

In one embodiment, the individual action correction module 5 shows the demo animation to the action correction object, wherein the individual action correction module 5 performs the following operations, including:

acquiring the face position of the action correction object and the screen center position of the teaching screen for training and teaching beside the action correction object respectively, wherein the face position can be determined by the personnel image of the action correction object;

determining the straight-line distance between the face position and the screen center position;

determining the display size requirement corresponding to the straight-line distance from the preset display size requirement library, wherein the display size requirement library has the display size requirements corresponding to different straight-line distances, and the display size requirement is the smallest size under the straight-line distance that the action correction object can view the content on the teaching screen;

determining multiple free display areas that meet the display size requirement from the teaching screen, wherein the teaching screen reserves some free display areas in advance for showing the demo animation;

acquiring the target face orientation of the action correction object;

constructing the first direction vector based on the face position and the target face orientation;

acquiring the directly faced orientation of the teaching screen;

constructing the second direction vector based on the region center position of the free display area and the directly faced orientation;

calculating the first vector angle between the first direction vector and the second direction vector;

suspending the demo animation on the free display area corresponding to the largest first vector angle to show, wherein the larger first vector angle means that the face of the action correction object is facing more directly the free display area, for example, when the action correction object completely directly faces the free display area, the first vector angle is 180 degrees.

The step of acquiring the target face orientation of the action correction object includes:

acquiring the current face orientation of the action correction object, wherein the face orientation can also be determined by the personnel image of the action correction object;

trying to acquire multiple desirable face directions of the action correction object in the future preset duration, wherein the preset duration can be, such as 4s, and the desirable face direction is a face orientation when the action correction object learns to follow the training action next, and can be determined according to actions to be taught in the future in the training teaching video;

taking the face orientation as the target face orientation when the try fails; otherwise, integrating the face orientation and the desirable face directions to acquire the face orientation set, wherein when the desirable face direction does not exist, it means that the currently learned training action will be taught continuously in the future preset duration; and it only needs to ensure that the angle of view between the current face orientation of the user and the free display area is appropriate, such that the face orientation is taken as the target face orientation;

constructing a third direction vector and a fourth direction vector respectively based on the face position and any two face orientations in the face orientation set, wherein the third direction vector is constructed based on the face position and one face orientation in any two face orientations, and the fourth direction vector is constructed based on the face position and the other face orientation in any two face orientations;

calculating the second vector angle between the third direction vector and the fourth direction vector; and

taking the direction of the sum vector of the third direction vector and the fourth direction vector of the largest second vector angle produced by calculation as the target face orientation, wherein when the second vector angle is maximum, it means that the corresponding two face orientations are the orientations that the face of the action correction object moves farthest in the future preset duration; and it only needs to ensure that the angle of view between the direction of the sum vector (i.e., the middle direction) of the third direction vector and the fourth direction vector and the free display area is appropriate, so that the face of the action correction object does not need to move more when viewing the demo animation and immediately doing the following training action.

The working principles and beneficial effects of the above technical solution are as follows.

When showing the demo animation to the action correction object, the embodiment of the present disclosure determines the free display area (i.e., the free display area corresponding to the maximum first vector angle) on the teaching screen that is most suitable for the action correction object, which improves the suitability of displaying the demo animation. Additionally, in general, when the individual correction moment is the repetition training progress, during the process of suspending the demo animation to show, the action correction object needs to instantly view the action and correct it, and further needs to perform the next follow action. When the action correction object views the free display area, the face direction will be changed. If the position at which the display area is viewed is unreasonable and the action correction object needs to change the face direction in the next follow action, the change degree of the face orientation of the action correction object in the next follow action needs to be greater, such that the action correction object may not be able to respond immediately, thereby failing to keep up with the training beat. Therefore, the embodiment of the present disclosure reasonably determines the target face orientation to ensure that this phenomenon does not occur as far as possible, which is more humanized, and more intelligent at the same time.

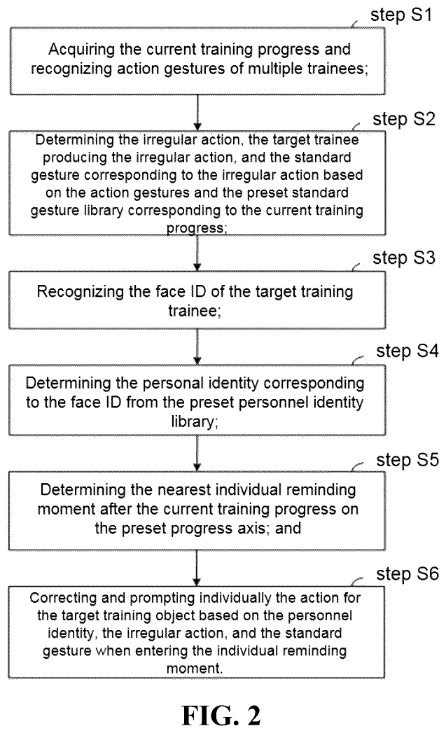

The group training action correction method combining face and gesture recognition provided by the embodiment of the present disclosure, as shown in FIG. 2, includes:

step S1: acquiring a current training progress and recognizing training gestures of multiple trainees;

step S2: determining whether the training gesture is standard based on the preset standard gesture corresponding to the current training progress, wherein, if not, the corresponding training gesture is taken as the action to be corrected and the trainee generating the action to be corrected is taken as the action correction object;

step S3: recognizing the face ID of the action correction object and acquiring the preset personnel identity corresponding to the face ID;

step S4: determining the nearest individual action correction moment after the current training progress on the preset training progress axis; and

step S5: correcting and prompting the individual action for the action correction object based on the standard gesture, the action to be corrected, and the personnel identity when entering the individual correction moment.

The step S4 of determining the nearest individual action correction moment after the current training progress on the preset training progress axis includes:

determining the action training cycle that the current training progress falls on the training progress axis;

determining the next action training cycle after the action training cycle on the training progress axis;

determining whether the next action training cycle is the same as the action training cycle,

determining the repetition training progress corresponding to the current training progress from the next action training cycle and taking as the individual action correction moment when they are the same; otherwise, determining whether the first gap time interval exists between the action training cycle and the next action training cycle,

taking the start moment of the first gap time interval as the individual action correction moment, if yes; otherwise, acquiring the correlation relationship between the action training cycle and the next action training cycle;

matching the correlation relationship with the triggered correlation relationship in the preset triggered correlation relationship library,

inserting the preset second gap time interval immediately after the end moment of the next action training cycle when the match exists; otherwise, inserting the second gap time interval immediately after the end moment of the action training cycle; and

taking the start moment of the second gap time interval as the individual action correction moment.

The step of correcting and prompting individually the actions for the action correction object based on the standard gesture, the action to be corrected, and the personnel identity includes:

acquiring the preset first virtual action corresponding to the standard gesture and the preset second virtual action corresponding to the action to be corrected respectively;

acquiring the action change process that the second virtual action changes to the first virtual action;

generating the demo animation for demonstrating the action change process;

acquiring the complexity of the action change process, the maximum reminder duration of the individual correction moment, and the training experience value of the action correction object respectively;

determining the play counts and the single-play duration of the demo animation based on the complexity, the maximum reminder duration, and the training experience value;

adjusting the animation duration of the demo animation to the single-play duration;

labeling the personnel identity in the demo animation; and

showing the demo animation to the action correction object, and controlling the play counts of continuously playing the demo animation when showing.

The step of determining play counts and the single-play duration of the demo animations based on the complexity, the maximum reminder duration, and the training experience value includes:

calculating the control value based on the complexity, the maximum reminder duration, and the training experience value, wherein the calculation formula is as follows:

$$ref = {γ}_{1} \cdot D + {γ}_{2} \cdot T + {γ}_{3} \cdot E$$

where ref is the control value, D is the complexity, T is the maximum reminder duration, E is the training experience value, and γ₁, γ₂, and γ₃ are the preset weight values;

acquiring the preset play count determination library, wherein the play count determination library includes multiple groups of one-to-one corresponding control value intervals and count terms;

determining whether the control value falls into any of the control value intervals;

taking the count term corresponding to a control value interval into which the control value falls as play counts, if yes; and

calculating the single-play duration based on the play counts and the maximum reminder duration, wherein the calculation formula is as follows:

$$t = \frac{T}{N}$$

where t is the single-play duration, T is the maximum reminder duration, and N is the play counts.

The step of showing the demo animation to the action correction object includes:

acquiring the face position of the action correction object and the screen center position of the teaching screen for training and teaching beside the action correction object respectively;

determining the straight-line distance between the face position and the screen center position;

determining the display size requirement corresponding to the straight-line distance from the preset display size requirement library;

determining multiple free display areas that meet the display size requirement from the teaching screen;

acquiring the target face orientation of the action correction object;

constructing the first direction vector based on the face position and the target face orientation;

acquiring the directly faced orientation of the teaching screen;

constructing the second direction vector based on the region center position of the free display area and the directly faced orientation;

calculating the first vector angle between the first direction vector and the second direction vector;

suspending the demo animation on the free display area corresponding to the largest first vector angle to show, wherein

the step of acquiring the target face orientation of the action correction object includes:

acquiring the current face orientation of the action correction object;

trying to acquire multiple desirable face directions of the action correction object in the future preset duration;

taking the face orientation as the target face orientation when the try fails; otherwise, integrating the face orientation and the desirable face directions to acquire the face orientation set;

constructing the third direction vector and the fourth direction vector respectively based on the face position and any two face orientations in the face orientation set;

calculating the second vector angle between the third direction vector and the fourth direction vector; and

taking the direction of the sum vector of the third direction vector and the fourth direction vector of the largest second vector angle produced by calculation as the target face orientation.

Obviously, those skilled in the art can make various modifications and variations of the present disclosure without departing from the spirit and scope of the present disclosure. Therefore, the present disclosure is intended to include these modifications and variations if these modifications and variations of the present disclosure fall within the scope of the claims of the present disclosure and their technical equivalents.

What is claimed is:

1. A group training action correction system combining face and gesture recognition, comprising:
a training gesture recognition module, configured to acquire a current training progress and recognize training gestures of multiple trainees;
a gesture specification determination module, configured to determine whether the training gestures are standard based on a preset standard gesture corresponding to the current training progress, wherein the corresponding training gestures are taken as actions to be corrected and the trainees generating the actions to be corrected are taken as action correction objects when the training gestures are not standard;
a personnel identity determination module, configured to recognize face IDs of the action correction objects and acquire preset personnel identities corresponding to the face IDs;
an individual action correction moment determination module, configured to determine a nearest individual action correction moment after the current training progress on a preset training progress axis; and
an individual action correction module, configured to correct and prompt individually the actions for the action correction objects based on the standard gesture, the actions to be corrected, and the personnel identities when entering the individual action correction moment, wherein
the individual action correction moment determination module determines the nearest individual action correction moment after the current training progress on the preset training progress axis, comprising:
determining an action training cycle that the current training progress falls on the training progress axis;
determining a next action training cycle after the action training cycle on the training progress axis;
determining whether the next action training cycle is the same as the action training cycle;
determining a repetition training progress corresponding to the current training progress from the next action training cycle and taking as the individual action correction moment when the next action training cycle is the same as the action training cycle; otherwise, determining whether a first gap time interval exists between the action training cycle and the next action training cycle;
taking a start moment of the first gap time interval as the individual action correction moment when the first gap time interval exists between the action training cycle and the next action training cycle; otherwise, acquiring a correlation relationship between the action training cycle and the next action training cycle;
matching the correlation relationship with a triggered correlation relationship in a preset triggered correlation relationship library;
inserting a preset second gap time interval immediately after an end moment of the next action training cycle when the match exists; otherwise, inserting the second gap time interval immediately after an end moment of the action training cycle; and
taking a start moment of the second gap time interval as the individual action correction moment.

2. The group training action correction system combining face and gesture recognition according to claim 1, wherein the individual action correction module corrects and prompts the individually the actions for the action correction objects based on the standard gesture, the actions to be corrected, and the personnel identities, comprising:
acquiring a preset first virtual action corresponding to the standard gesture and a preset second virtual action corresponding to the actions to be corrected respectively;
acquiring an action change process that the second virtual action changes to the first virtual action;
generating a demo animation for demonstrating the action change process;
acquiring a complexity of the action change process, a maximum reminder duration of the individual action correction moment, and a training experience value of the action correction objects respectively;
determining play counts and a single-play duration of the demo animation based on the complexity, the maximum reminder duration, and the training experience value;
adjusting an animation duration of the demo animation to the single-play duration;
labeling the personnel identities in the demo animation; and
showing the demo animation to the action correction objects, and controlling the play counts of continuously playing the demo animation when showing.

3. The group training action correction system combining face and gesture recognition according to claim 2, wherein the individual action correction module determines the play counts and the single-play duration of the demo animation based on the complexity, the maximum reminder duration, and the training experience value, comprising:
calculating a control value based on the complexity, the maximum reminder duration, and the training experience value, wherein a calculation formula is as follows:
$$ref = {γ}_{1} \cdot D + {γ}_{2} \cdot T + {γ}_{3} \cdot E$$
where ref is the control value, D is the complexity, T is the maximum reminder duration, E is the training experience value, and γ₁, γ₂ and γ₃ are preset weight values;
acquiring a preset play count determination library, wherein the play count determination library comprises multiple groups of one-to-one corresponding control value intervals and count terms;
determining whether the control value falls into any of the control value intervals;
taking the count terms corresponding to the control value intervals into which the control value falls as the play counts when the control value falls into the control value intervals; and
calculating the single-play duration based on the play counts and the maximum reminder duration, wherein a calculation formula is as follows:
$$t = \frac{T}{N}$$
where t is the single-play duration, T is the maximum reminder duration, and N is the play counts.

4. The group training action correction system combining face and gesture recognition according to claim 2, wherein the individual action correction module shows the demo animation to the action correction objects, comprising:
acquiring face positions of the action correction objects and a screen center position of a teaching screen for training and teaching beside the action correction objects respectively;
determining a straight-line distance between the face positions and the screen center position;
determining a display size requirement corresponding to the straight-line distance from a preset display size requirement library;
determining multiple free display areas that meet the display size requirement from the teaching screen;
acquiring a target face orientation of the action correction objects;
constructing a first direction vector based on the face positions and the target face orientation;
acquiring a directly faced orientation of the teaching screen;
constructing a second direction vector based on a region center position of the free display areas and the directly faced orientation;
calculating a first vector angle between the first direction vector and the second direction vector; and
suspending the demo animation on a free display area corresponding to a largest first vector angle to show, wherein
the step of acquiring a target face orientation of the action correction objects comprises:
acquiring current face orientations of the action correction objects;
trying to acquire multiple desirable face directions of the action correction objects in a future preset duration;
taking the face orientations as the target face orientation when the try fails; otherwise, integrating the face orientations and the desirable face directions to acquire a face orientation set;
constructing a third direction vector and a fourth direction vector respectively based on the face positions and any two face orientations in the face orientation set;
calculating a second vector angle between the third direction vector and the fourth direction vector; and
taking a direction of a sum vector of the third direction vector and the fourth direction vector of a largest second vector angle produced by calculation as the target face orientation.

5. A group training action correction method combining face and gesture recognition, comprising:
step S1: acquiring a current training progress and recognizing training gestures of multiple trainees;
step S2: determining whether the training gestures are standard based on a preset standard gesture corresponding to the current training progress, wherein the corresponding training gestures are taken as actions to be corrected and the trainees generating the actions to be corrected are taken as action correction objects when the training gestures are not standard;
step S3: recognizing face IDs of the action correction objects and acquiring preset personnel identities corresponding to the face IDs;
step S4: determining a nearest individual action correction moment after the current training progress on a preset training progress axis; and
step S5: correcting and prompting individually actions for the action correction objects based on the standard gesture, the actions to be corrected, and the personnel identities when entering the individual action correction moment, wherein
the step S4 of determining a nearest individual action correction moment after the current training progress on a preset training progress axis comprises:
determining an action training cycle that the current training progress falls on the training progress axis;
determining a next action training cycle after the action training cycle on the training progress axis;
determining whether the next action training cycle is the same as the action training cycle;
determining a repetition training progress corresponding to the current training progress from the next action training cycle and taking as the individual action correction moment when the next action training cycle is the same as the action training cycle; otherwise, determining whether a first gap time interval exists between the action training cycle and the next action training cycle;
taking a start moment of the first gap time interval as the individual action correction moment when the first gap time interval exists between the action training cycle and the next action training cycle; otherwise, acquiring a correlation relationship between the action training cycle and the next action training cycle;
matching the correlation relationship with a triggered correlation relationship in a preset triggered correlation relationship library;
inserting a preset second gap time interval immediately after an end moment of the next action training cycle when the match exists; otherwise, inserting the second gap time interval immediately after an end moment of the action training cycle; and
taking a start moment of the second gap time interval as the individual action correction moment.

6. The group training action correction method combining face and gesture recognition according to claim 5, wherein the step of correcting and prompting individually actions for the action correction objects based on the standard gesture, the actions to be corrected, and the personnel identities comprises:
acquiring a preset first virtual action corresponding to the standard gesture and a preset second virtual action corresponding to the actions to be corrected respectively;
acquiring an action change process that the second virtual action changes to the first virtual action;
generating a demo animation for demonstrating the action change process;
acquiring a complexity of the action change process, a maximum reminder duration of the individual action correction moment, and a training experience value of the action correction objects respectively;
determining play counts and a single-play duration of the demo animation based on the complexity, the maximum reminder duration, and the training experience value;
adjusting an animation duration of the demo animation to the single-play duration;
labeling the personnel identities in the demo animation; and
showing the demo animation to the action correction objects, and controlling the play counts of continuously playing the demo animation when showing.

7. The group training action correction method combining face and gesture recognition according to claim 6, wherein the step of determining play counts and a single-play duration of the demo animation based on the complexity, the maximum reminder duration, and the training experience value comprises:
calculating a control value based on the complexity, the maximum reminder duration, and the training experience value, wherein a calculation formula is as follows:
$$ref = {γ}_{1} \cdot D + {γ}_{2} \cdot T + {γ}_{3} \cdot E$$
where ref is the control value, D is the complexity, T is the maximum reminder duration, E is the training experience value, and γ₁, γ₂ and γ₃ are preset weight values;
acquiring a preset play count determination library, wherein the play count determination library comprises multiple groups of one-to-one corresponding control value intervals and count terms;
determining whether the control value falls into any of the control value intervals;
taking the count terms corresponding to the control value intervals into which the control value falls as the play counts when the control value falls into the control value intervals; and
calculating the single-play duration based on the play counts and the maximum reminder duration, wherein a calculation formula is as follows:
$$t = \frac{T}{N}$$
where t is the single-play duration, T is the maximum reminder duration, and N is the play counts.

8. The group training action correction method combining face and gesture recognition according to claim 6, wherein the step of showing the demo animation to the action correction objects comprises:
acquiring face positions of the action correction objects and a screen center position of a teaching screen for training and teaching beside the action correction objects respectively;
determining a straight-line distance between the face positions and the screen center position;
determining a display size requirement corresponding to the straight-line distance from a preset display size requirement library;
determining multiple free display areas that meet the display size requirement from the teaching screen;
acquiring a target face orientation of the action correction objects;
constructing a first direction vector based on the face positions and the target face orientation;
acquiring a directly faced orientation of the teaching screen;
constructing a second direction vector based on a region center position of the free display areas and the directly faced orientation;
calculating a first vector angle between the first direction vector and the second direction vector; and
suspending the demo animation on a free display area corresponding to the largest first vector angle to show, wherein
the step of acquiring a target face orientation of the action correction objects comprises:
acquiring current face orientations of the action correction objects;
trying to acquire multiple desirable face directions of the action correction objects in a future preset duration;
taking the face orientations as the target face orientation when the try fails; otherwise, integrating the face orientations and the desirable face directions to acquire a face orientation set;
constructing a third direction vector and a fourth direction vector respectively based on the face positions and any two face orientations in the face orientation set;
calculating a second vector angle between the third direction vector and the fourth direction vector; and
taking a direction of a sum vector of the third direction vector and the fourth direction vector of a largest second vector angle produced by calculation as the target face orientation.
